(19) United States
(12) Patent Application Publication
(10) Pub. No.: US 2020/0377932 A1
(43) Pub. Date: Dec. 3, 2020

(54) DDX24 MUTATIONS AND USE THEREOF

(71) Applicant: The Fifth Affiliated Hospital of Sun Yat-Sen University, Guangdong (CN)

(72) Inventors: Hong SHAN, Guangdong (CN); Pengfei PANG, Guangdong (CN); Xiaojun HU, Guangdong (CN); Junjie MAO, Guangdong (CN)

(21) Appl. No.: 16/631,370

(22) PCT Filed: Dec. 12, 2017
(86) PCT No.: PCT/CN2017/117951

§ 371 Date: Jan. 15, 2020

(30) Foreign Application Priority Data
Dec. 13, 2017 (CN) 2017113270591

(51) Int. Cl. C12Q 1/6827; C12Q 1/6844

(52) U.S. Cl. CPC C12Q 1/6827; A61K 45/06; C12Q 2600/112; C12Q 1/6844

(57) ABSTRACT

Provided are DDX24 mutations and the use thereof, wherein the mutations include Glu271Lys, Lys11Glu and Arg436His. The mutations, i.e. Glu271Lys, Lys11Glu and Arg436His, of DDX24 gene are significantly associated with the development of blood vessels. It would result in vascular malformations by interfering with DDX24. In addition, the onset of human vascular malformations can be predicted by detecting the SNP sites of DDX24.

### CROSS-REFERENCE TO RELATED APPLICATIONS

This application is based upon and claims priority to Chinese Patent Application No. 2017113270591, filed on Dec. 13, 2017, the entire contents of which are incorporated herein by reference.

### TECHNICAL FIELD

The present disclosure belongs to the field of molecular biology, and relates to novel gene mutations and new use thereof.

### BACKGROUND

In liver-related diseases, the malformation of portal and hepatic veins can lead to serious health problems, among which Cavernous Transformation of Portal Vein (CTPV) and Budd-Chiari syndrome (BCS) have relatively high incidences. The common features shared by CTPV and BCS are vein stenosis or occlusion with formation of collateral circulation. The causes of CTPV and BCS comprise local and systemic factors, such as trauma, inflammation, external stress, hemodynamic changes and hypercoagulable state. However, no explicit pathogenesis has been determined for some patients with CTPV or BCS. Most of these patients have poor prognosis due to lack of accurate diagnosis and reasonable treatment. It has been reported that some gene mutations, such as mutations in JAK2, are associated with BCS, but no pathogenic gene has been reported to be related to CTPV.

DEAD-box helicase 24 (DDX24) is a member of the helicase gene family, located in Region 32 on the long arm of chromosome 14, and widely distributed in various tissues of human bodies. Similar to other helicase genes, DDX24 plays an important role in gene repair, and is closely related to unstable inheritance. Studies have shown that DDX24 is associated with malignant tumors. However, it has not been reported that DDX24 is associated with vascular malformation.

### SUMMARY

One object of the present disclosure is intended to provide novel DDX24 mutations, and the use thereof.

The technical solutions adopted by the present disclosure are as follows.

One aspect of the present disclosure provides Single Nucleotide Polymorphism (SNP) mutations of DDX24 gene, which may include Glu271Lys, Lys11Glu and Arg436His.

Another aspect of the present disclosure provides the use of the SNP mutations of DDX24 gene as a marker for detecting vascular malformation.

The vascular malformation may include CTPV, BCS, and refractory chylothorax caused by thoracic duct obliteration.

Still another aspect of the present disclosure provides the use of a reagent for detecting the SNP mutation of DDX24 gene in the preparation of a screening reagent for the vascular malformation.

Further, the reagent for detecting the SNP mutation of DDX24 gene may include a gene amplification reagent, a gene sequencing reagent, and a protein sequence analytical reagent.

Still another aspect of the present disclosure provides the use of a reagent for repairing the SNP mutation of DDX24 gene in the preparation of a gene therapeutic medicament for treating the vascular malformation.

The present disclosure may have the following beneficial effects.

The mutations, i.e. Glu271Lys, Lys11Glu and Arg436His, of DDX24 gene are significantly associated with the development of vessels. Cell migration and vasculogenesis, which may suggest the development of vascular malformation, can be promoted by interfering DDX24. The detection of the SNP sites of DDX24 can be used to effectively predict the development of vascular malformation in human body, to screen genetic defects, and to correctly determine the type of vessel abnormalities, thereby reducing misdiagnosis and facilitating targeted therapy.

### BRIEF DESCRIPTION OF THE DRAWINGS

FIG. 1 shows pedigree of an family with inherited CTPV and the incidence thereof.

FIGS. 2 to 8 show the clinical and histological images or photographs of the patients from the CTPV family;

FIG. 9 shows different mutation sites and a protein model of DDX24;

FIG. 10 shows a result obtained from conservation analysis of DDX24;

FIG. 11 shows an alignment result between DDX24 and HERA;

FIG. 12 shows the influence results of siRNA-mediated knockdown of DDX24 in HUVECs; and

FIG. 13 shows the influence results of siRNA-mediated knockdown of DDX24 on the growth of HUVECs.

### DETAILED DESCRIPTION

The inventors evaluated a four-generation family with inherited CTPV and hepatic vein stenosis. In this family, some members with CTPV and hepatic vein stenosis also have other disorders, including chylothorax and pulmonary valve stenosis. The inventors performed intensive research and analysis in order to determine the genetic basis of this comprehensive disease and other similar phenotypes, and to further analyze the pathophysiological basis thereof.

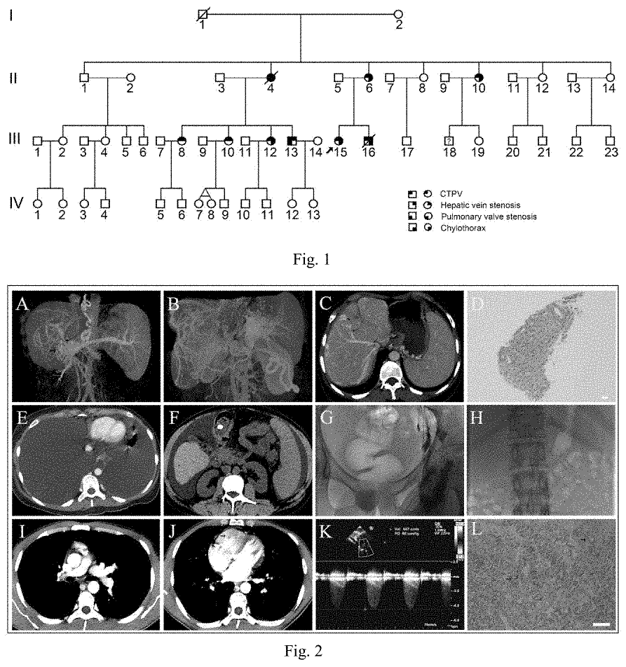

The proband of the family was noticed by the inventors due to refractory chylothorax. After clinical investigation of the proband, the inventors collected clinical information of all the members from the family, which comprises a total of 52 members, across four generations, and has nine affected members. Seven alive patients were enrolled in the study (FIG. 1). The medical records, including imaging records, of two deceased members from the family were also studied at the same time. Also enrolled in the study were 10 patients with sporadic congenital CTPV and 151 patients with congenital BCS. All the participants were of Chinese Han ethnicity, and had signed written informed consent. The study was approved by Ethics Committee of the Fifth Affiliated Hospital of Sun Yat-sen University, following the principles of Helsinki Declaration.

### Diagnosis and Clinical Evaluation

The inventors examined the medical records, including the imaging records of two deceased members from the family. In addition, the subjects who were ≥13 years old at the time of enrollment, were confirmed suffering CTPV and hepatic vein stenosis by using computed tomography (CT), and the subjects who were <13 years old were examined with ultrasonography. These examinations were performed by three independent imaging specialists. Three affected members (II-6, III-8, and III-16) were examined with liver biopsies. The inventors explored all possible causes of CTPV and hepatic vein stenosis the family. The sporadic congenital CTPV or BCS was diagnosed and classified according to “AASLD Practice Guidelines and EASL Clinical Practice Guidelines for vascular disease of the liver”.

### Genetic Analysis

Chromosomal microarray analysis using Agilent CytoGenomics software (version 3.0. http://www.genomics.agilent.com/) was performed for the DNA samples from the family members (II-8, II-10, II-12 and III-15), to determine whether any change at the chromosome level was responsible for the disease phenotypes. The data was analyzed by using Online Mendelian Inheritance in Man (OMIM, http://omim.org/), UniGene (https://www.ncbi.nlm.nih.gov/unigene), Conserved Domain Database (https://www.ncbi.nlm.nih.gov/Structure/cdd/cdd.shtml), and BioGPS (http://biogps.org/). Polymorphic copy number variations, which have been reported in Database of Genomic Variants 14 (http://dgv.tcag.ca/dgv/app/home), was excluded from further consideration.

The genomic DNA of all available members from the family was genotyped by using SNP Array 6.0 (Affymetrix) covering 908,476 SNP markers. LOD scores were calculated by using MERLIN 1.1.2.2. Linkage analysis identified candidate regions which had LOD scores >3.0. In the candidate regions, one of every 5 SNPs was selected as a tag-SNP, and haplotypes were constructed by using HaploPainter. For co-segregation haplotype analysis, SNP pathogenicity was classified according to American Society of Medical Genetics and Genomics (ACMG) guidelines.

Whole exome sequencing was performed for two affected (III-8 and III-10) and two unaffected (II-8 and II-12) members from the CTPV family. The exome sequencing was performed h using Illumina Hiseq2000 with a paired-end 100-bp length configuration. High-quality reads were mapped against the Human Reference Genome (hg1.9) from the University of California, Santa Cruz (UCSC) Genome Browser (http://genome.ucsc.edu/) by using Burrows-Wheeler Aligner (BWA) program. SNPs were identified by using SOAP snp (version 0.1.19, https://sourceforge.net/projects/soapsnp/files/soapsnp/download), and small insertions/deletions were identified by using Samtools. Variants were annotated by using ANNOVAR (Supplementary Appendix). All variants were predicted according to Polyphen2 (http://genetics.bwh.harvard.edu/pph2/). The variants, which were shared by 2 affected members but absent in 2 unaffected members of the family, were further analyzed.

Sanger sequencing was used to further confirm the mutations which were shared by 2 affected members and absent in 2 unaffected members of the family.

The analysis results showed that DDX24 gene is a pathogenic gene. Subsequently, DNA sequencing was performed for the exon-intron boundaries of DDX24 gene in 10 patients with sporadic congenital CTPV and 151 patients with congenital BCS.

### Cell Function Assays

Two siRNAs (siRNA#1: 5′-GCAGUCAAGCUGUGGCAAA-3′; siRNA#2: 5′-CCUGUAAGGCAUAUCCAAA-3′) targeting human DDX24 gene were designed by using GenScript bioinformatics tools (https://www.genscript.com/tools/sirna-target-finder), and transfected into Human Umbilical Vein Endothelial Cells (HUVECs) by using Lipofectamine 3000 (Invitrogen). The control was transfected with scramble siRNA (RIBOBIO). The siRNA-interfering efficiency was determined by using western blotting. Cell viability was evaluated by using a Cell Counting Kit (CCK)-8 kit (Dojinbo Molecular Technologies). Cell migration was examined by using a modified 24-well traswell assay. A tube formation assay was performed in accordance with existing protocols. RNA sequencing analysis was performed with siRNA-targeted cells, to evaluate the influence of knockdown DDX24 on gene expression.

### Structural Model

Discovery studio 3.5 (BIOVIA) was used to construct a structural model of a ATP binding region of DDX24. The crystal structure of the N-terminal domain of HERA (Protein Data Bank (PDB) ID: 2GXQ) was used as a template for homology modeling. MODELER was used to evaluate the structural model of the ATP binding region of DDX24.

### Results

Case Summary

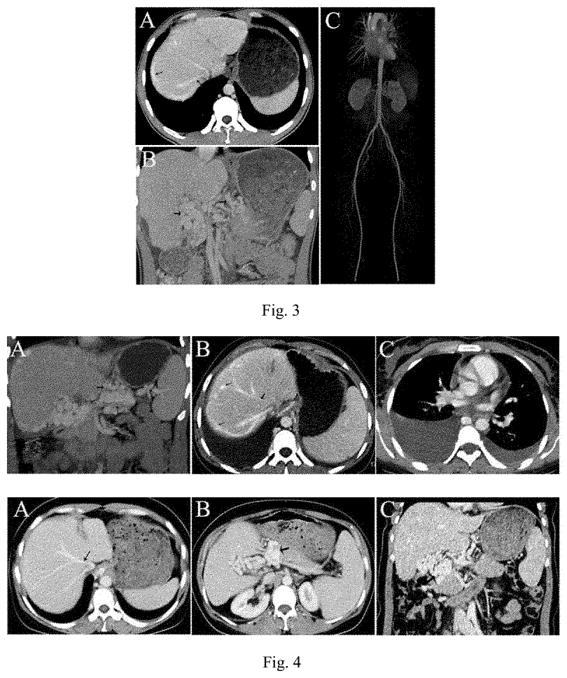

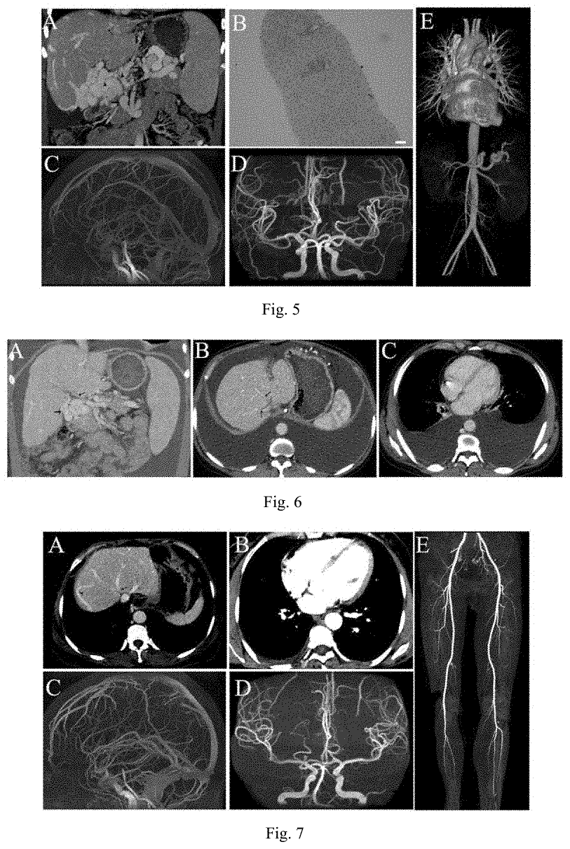

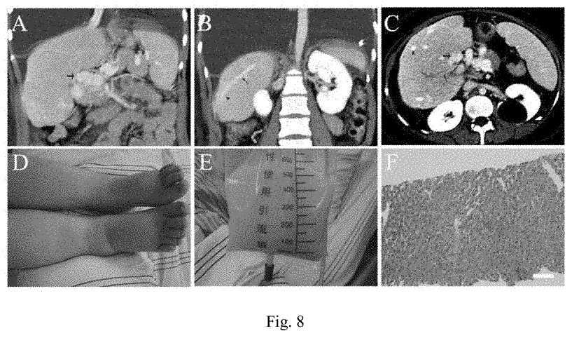

All the affected members from the large family had CTPV and hepatic vein stenosis. The clinical imaging and histological characteristics were shown in FIGS. 2-8. The proband (III-15) as well as her younger brother (III-16) and aunt (II-4) were hospitalized due to refractory chylothorax caused by thoracic duct obliteration, the latter two died of respiratory failure later. Other family members III-6, II-10 and III-12 also had moderate chylothorax. II-4 and III-13 had pulmonary valve stenosis. II-10, III-12 and III-15 had mild pericardial effusion. However, the nature of the effusion was not determined because the aspiration assay was not performed. II-10 and III-15 also had ascites. Other affected members from the family than III-15 and III-13 were observed to have splenomegaly and oesophageal varices. III-8, III-12 and III-15 had fundus varication. III-8 had a gastro-renal shunt, and II-6 had portal venous aneurysm. All members had no gastrointestinal bleeding history. No abnormal member was found by ultrasonography in the fourth generation (0.5 to 11 years old). The phenotypes of the affected members from the family and of the sporadic congenital CTPV and BCS patients are shown in Table 1.

TABLE 1
Clinical characteristics of the patients and the DDX24 mutations thereof

| | | | | |
|---|---|---|---|---|
| Patients | Gender* | Age | Clinical characteristics | Mutations |

Patients of CPTV family

| | | | | |
|---|---|---|---|---|
| II-4 | F | 43 | CTPV, hepatic vein stenosis, chylothorax, | ND^{#} |
| | | | pulmonary valve stenosis, splenomegaly, | |
| | | | oesophageal varices | |
| II-6 | F | 50 | CTPV, hepatic vein stenosis, chylothorax, | p.Glu271Lys |
| | | | splenomegaly, oesophageal varices, | |
| | | | portal cavernoma | |
| II-10 | F | 44 | CTPV, hepatic vein stenosis, chylothorax, | p.Glu271Lys |
| | | | ascites, pericardial effusion, splenomegaly, | |
| | | | oesophageal varices | |
| III-8 | F | 36 | CTPV, hepatic vein stenosis, splenomegaly, | p.Glu271Lys |
| | | | oesophageal varices, fundus varication, | |
| | | | gastro-renal shunt | |
| III-10 | F | 31 | CTPV, hepatic vein stenosis, splenomegaly, | p.Glu271Lys |
| | | | oesophageal varices | |
| III-12 | F | 28 | CTPV, hepatic vein stenosis, chylothorax, | p.Glu271Lys |
| | | | pericardial effusion, splenomegaly, | |
| | | | oesophageal varices, fundus varication | |
| III-13 | M | 27 | CTPV, hepatic vein stenosis, pulmonary valve | p.Glu271Lys |
| | | | stenosis, splenomegaly | |
| III-15 | F | 16 | CTPV, hepatic vein stenosis, chylothorax, | p.Glu271Lys |
| | | | ascites, pericardial effusion, oesophageal | |
| | | | varices, fundus varication | |
| II-16 | F | 14 | CTPV, hepatic vein stenosis, chylothorax, | ND |
| | | | splenomegaly, oesophageal varices | |

Sporadic CTPV patient

| | | | | |
|---|---|---|---|---|
| GD01 | M | 43 | CTPV | p.Glu271Lys |

Budd-Chiari syndrome (BCS) patients

| | | | Diseased | Mutation |
|---|---|---|---|---|
| Patient | Gender* | Age | region | sites |
| XZ012 | F | 32 | HV^{f} | p.Glu271Lys |
| XZ015 | M | 46 | IVC″ | p.Glu271Lys |
| XZ022 | F | 38 | HV | p.Glu271Lys |
| XZ028 | M | 43 | IVC | p.Glu271Lys |
| XZ030 | M | 50 | IVC | p.Glu271Lys |
| XZ049 | F | 60 | HV + IVC | p.Glu271Lys |
| XZ052 | M | 30 | IVC | p.Glu271Lys |
| XZ057 | M | 21 | HV | p.Glu271Lys |
| XZ061 | M | 24 | HV + IVC | p.Glu271Lys |
| XZ062 | F | 47 | IVC | p.Glu271Lys |
| XZ066 | F | 29 | HV | p.Glu271Lys |
| XZ069 | M | 50 | IVC | p.Glu271Lys |
| XZ101 | M | 65 | IVC | p.Glu271Lys |
| XZ100 | M | 31 | HV | p.Glu271Lys |
| XZ003 | F | 23 | HV | p.LysllGlu |
| XZ004 | F | 45 | HV | p.LysllGlu |
| XZ106 | F | 45 | IVC | p.LysllGlu |
| XZ110 | M | 37 | IVC | p.LysllGlu |
| XZ113 | M | 60 | IVC | p.LysllGlu |
| XZ122 | F | 76 | IVC | p.LysllGlu |
| XZ130 | M | 21 | HV | p.LysllGlu |
| XZ132 | F | 60 | IVC | p.LysllGlu |
| XZ136 | F | 63 | IVC | p.LysllGlu |
| XZ142 | F | 39 | HV | p.LysllGlu |
| XZ103 | F | 41 | HV + IVC | p. Arg436His |

### Genetic Analysis

The results of chromosomal microarray analysis did not identify any genomic imbalance, such as a gain/loss of whole or part of chromosomes in the family members. This suggests that a small sequence variant(s), rather than large-scale genetic recombination, may be responsible for the clinical phenotypes in the family members.

Since the pedigree study had indicated that the phenotypes were inherited in an autosomal dominant fashion, the inventors performed a linkage analysis to identify the gene. The results showed that chromosome 14q32.12 had the highest ExLOD score of 3.59. A haplotype was identified by haplotype SNP analysis to have phenotypic co-segregation with the above family disease. This haplotype is shared by, all the affected members but III-18 from the family. III-18 was 13 years old when being enrolled in the study, and carried the pathogenic gene but had no identified clinical symptom. This may be due to the late onset of the disease or incomplete penetrance.

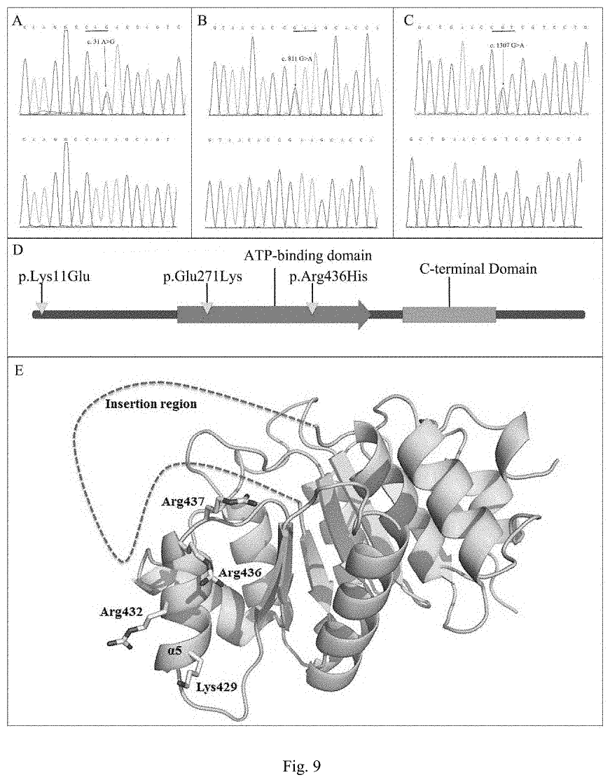

The sequencing results showed that 35 variants, which were shared by 2 patients but absent for the unaffected members of the family, in 23 genes were deleterious. Among the variants, Glu271Lys (rs149296999) in DDX24 was the only mutation identified in both the linked and co-segregated haplotypes. This mutation is a substitution mutation in which an alkaline lysine residue is substituted with an acidic glutamine residue, and is classified as a “likely pathogenic ” according to the ACMG guidelines. This mutation was also found in one sporadic congenital CTPV case (FIG. 9B).

### Congenital BCS and DDX24 Mutations

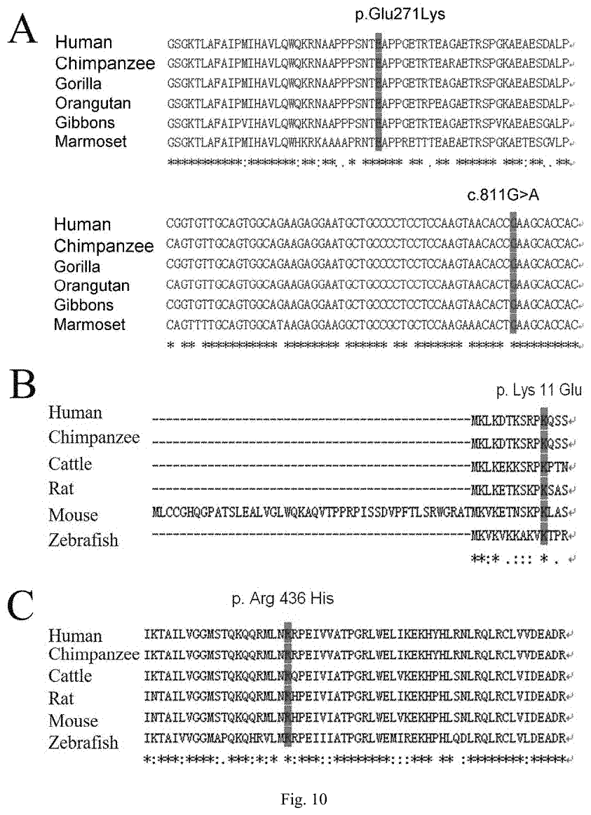

The mutation Glu271Lys of in DDX24 was identified in 14 cases from 151 patients with congenital BCS. Two further mutations in DDX24 were identified in the congenital BCS patients by Sanger sequencing and are classified as “pathogenic” or “likely pathogenic” according to the ACMG guidelines. A mutation Lys11Glu (rs142609376) was found in 10 BCS patients (FIG. 9A), and a mutation Arg436His was found in 1 patient (FIG. 9C). The 1000 Genomes Project showed that the mutations Glu271Lys and Lys11Glu had a 1% allele frequency in China's population, while the mutation Arg436His was not found. All the mutations identified in the patients are present in evolutionarily conserved sequences of DDX24 (FIG. 10), suggesting that the mutation-affected regions are essential for the function of the gene.

### Protein Modeling

The DDX24 protein was predicted to contain two domains: a helicase adenosine triphosphate (ATP)-binding domain (also known as the N-terminal domain of the homologous protein, residues 192-532) and a C-terminal domain (residues 578-732). Both Glu271 and Arg436 are located in the predicted ATP-binding domain (FIG. 9D). Therefore, the inventors constructed a structural model of the ATP-binding domain of DDX24 protein by using the N-terminal domain of HERA (Protein Data Bank (PDB) ID: 2GXQ) as a homologous template, to investigate how the Arg436His mutation affected the function of DDX24 (FIG. 9E).

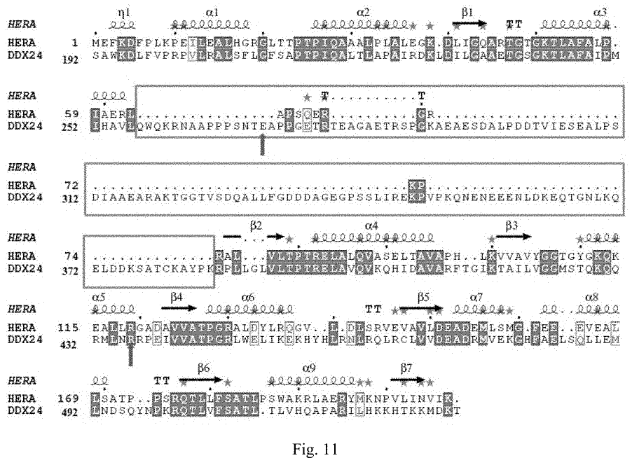

In the constructed model, Arg436 was located in the α5 helix and prone to be exposed in solvent. There was no significant interaction between Arg436 and the nearby residues. Based on this model, Arg436His mutation was unlikely to affect the overall structure of the ATP-binding domain of DDX24. However, it was found that the ATP-binding domain of DDX24 contained an additional insertion region of residues 257-385 (FIG. 11) through alignment between the N-terminal sequences of HERA and DDX24. This insertion region was close to the α5 helix. Alkaline Arg436 might affect the insertion region of the ATP-binding domain of DDX24, because the insertion region contained a large amount of negatively charged residues. Interestingly, in addition to Arg436, the α5 helix further contained alkaline Lys429, Arg432 and Arg437, which together created a positively charged region that might interact with a partner molecule (e.g. protein or RNA).

### Function Study

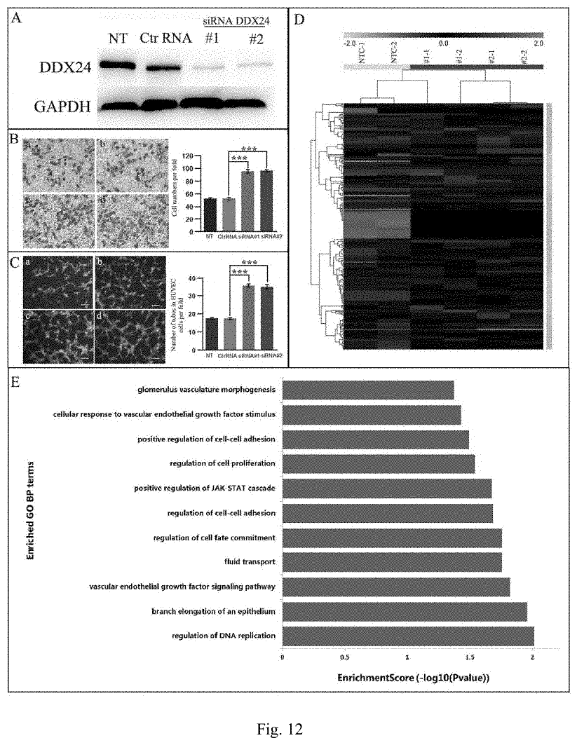

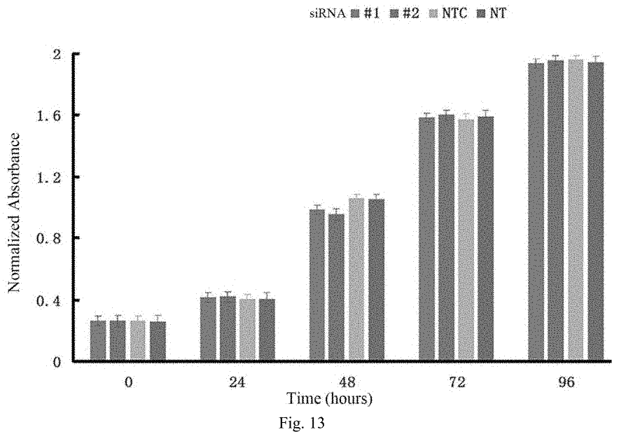

Genetic knockout of DDX24 has been report to lead to embryonic lethality. In order to investigate the function of DDX24, the inventors performed siRNA-mediated gene interference experiments in umbilical vein endothelial cells (HUVECs) (FIG. 12A). Although siRNA treatment did not affect the growth of HUVECs (FIG. 13), cell migration and tube formation were significantly enhanced with siRNA treatment, as compared to the control which was treated with scramble siRNA (FIG. 12B, C). It was revealed by heatmap analysis that 144 genes were up-regulated and 227 genes were down-regulated in HUVECs treated with DDX24 siRNA (FIG. 12D). Gene ontology (GO) analysis revealed that a group of genes having differential expression in the DDX24-knockdown cells were enriched in the vascular endothelial growth factor signaling pathway.

In summary, these results suggest that, the mutations Glu271Lys, Lys11Glu and Arg436His of DDX24 are significantly associated with vessel development. DDX24 knockdown can promote the cell migration and tube formation, resulting in vascular malformations. The SNP sites of DDX24 can be detected to effectively predict the vascular malformations in human body, to screen out genetic defects, and to facilitate correct determination of the types of vascular abnormalities, thereby reducing misdiagnosis and facilitating targeted therapy.

What is claimed is:

1. A Single Nucleotide Polymorphism (SNP) mutation in DDX24 gene, comprising Glu271Lys, Lys11Glu or Arg436His.

2. Use of a Single Nucleotide Polymorphism (SNP) mutation in DDX24 gene as defined in claim 1 as a marker for detecting vascular malformation.

3. The use according to claim 2, wherein the vascular malformation comprises Cavernous Transformation of Portal Vein (CTPV), Budd-Chiari syndrome (BCS), and refractory chylothorax caused by thoracic duct obliteration.

4. Use of a reagent for detecting a Single Nucleotide Polymorphism (SNP) mutation in DDX24 gene in the preparation of a screening reagent for vascular malformation, wherein the SNP mutation in DDX24 gene is as defined in claim 1.

5. The use according to claim 4, wherein the reagent for detecting the SNP mutation in DDX24 gene is selected from a group consisting of a gene amplification reagent, a gene sequencing reagent, and a protein sequence analytical reagent.

6. Use of a reagent for repairing a Single Nucleotide Polymorphism (SNP) mutation in DDX24 gene in the preparation of a gene therapeutic medicament for treating vascular malformation.

7. The use according to claim 6, wherein the vascular malformation comprises Cavernous Transformation of Portal Vein (CTPV), Budd-Chiari syndrome (BCS), and refractory chylothorax caused by thoracic duct obliteration.
